(19) United States
(12) Patent Application Publication
(10) Pub. No.: US 2026/0142963 A1
(43) Pub. Date: May 21, 2026

(54) MEDICAL DEVICE CERTIFICATE GATEWAY

(71) Applicant: Welch Allyn, Inc., Skaneateles Falls, NY (US)

(72) Inventors: Thomas Henry Briggs, Frederick, MD (US); Avinash Ashok Kank, Syracuse, NY (US); Steven D. Morrow, Vestal, NY (US); Derek Strassle, Port Byron, NY (US)

(21) Appl. No.: 19/389,561

(22) Filed: Nov. 14, 2025

Related U.S. Application Data
(60) Provisional application No. 63/722,700, filed on Nov. 20, 2024.

(51) Int. Cl. H04L 9/40; G16H 40/67

(52) U.S. Cl. CPC H04L 63/0823; G16H 40/67

(57) ABSTRACT

An authentication system includes a medical device, and a certificate bridge device configured to receive a request from the medical device. The certificate bridge device generates an endpoint request that includes the request from the medical device and facility credentials of a facility where the medical device is deployed. The certificate bridge device sends the endpoint request to a certificate authority, receives a signed copy of the request from the certificate authority, and sends the signed copy of the request to the medical device.

### CROSS-REFERENCE TO RELATED APPLICATION(S)

This application claims the benefit of U.S. Provisional Application No. 63/722,700, filed Nov. 20, 2024, the disclosure of which is hereby incorporated by reference in its entirety.

### BACKGROUND

Medical device authentication and security are critical components in safeguarding patient data and ensuring the integrity of healthcare systems. With the increasing connectivity of medical devices, including wearables and implanted devices, there is a heightened risk of cyber threats. Effective authentication mechanisms, such as multi-factor authentication and encryption, help verify the identity of users and devices, preventing unauthorized access.

Moreover, robust security measures are essential to protect sensitive patient information from breaches and attacks. Compliance with regulatory standards, like those set by the U.S. Food and Drug Administration (FDA) and Health Insurance Portability and Accountability Act (HIPAA), is vital in ensuring that medical devices are designed with security in mind. Ongoing monitoring, regular updates, and vulnerability assessments are also crucial to mitigate risks and maintain the reliability of medical devices throughout their lifecycle. Overall, authentication and security is essential for maintaining trust and safety in modern healthcare.

### SUMMARY

In general terms, the present disclosure relates to managing digital certificates for a medical devices within a facility. In one possible configuration, a certificate bridge device is utilized as an intermediary between the medical devices and one or more certificate authorities for the management and procurement of digital certificates. Various aspects are described in this disclosure, which include, but are not limited to, the following aspects.

One aspect relates to a device for managing digital certificates for a medical device, the device comprising: at least one processing device; and at least one memory device storing software instructions that, when executed by the at least one processing device, cause the at least one processing device to: receive a request from the medical device; authenticate the medical device; determine the medical device is authorized to send the request; validate the request from the medical device; build an endpoint request that includes: the request from the medical device; and facility credentials of a facility where the medical device is deployed; send the endpoint request to a certificate authority; receive a signed copy of the request from the certificate authority; and send the signed copy of the request to the medical device.

Another aspect relates to a method of managing digital certificates for a medical device, the method comprising: receiving a request from the medical device; authenticating the medical device; determining the medical device is authorized to send the request; validating the request from the medical device; building an endpoint request that includes: the request from the medical device; and facility credentials of a facility where the medical device is deployed; sending the endpoint request to a certificate authority; receiving a signed copy of the request from the certificate authority; and sending the signed copy of the request to the medical device.

Another aspect relates to an authentication system comprising: a medical device; and a certificate bridge device configured to: receive a request from the medical device; generate an endpoint request that includes the request from the medical device and facility credentials of a facility where the medical device is deployed; send the endpoint request to a certificate authority; receive a signed copy of the request from the certificate authority; and send the signed copy of the request to the medical device.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of a medical device that can be used to monitor one or more physiological variables of a patient.

FIG. 2 is a rear isometric view of the medical device of FIG. 1.

FIG. 3 is a front view of the medical device of FIG. 1.

FIG. 4 is a bottom view of the medical device of FIG. 1.

FIG. 5 schematically illustrates an example of the medical device of FIG. 1.

FIG. 6 schematically illustrates an example of a certificate bridge device that facilitates digital certificate management for a plurality of devices within a facility, including the medical device of FIG. 1.

FIG. 7 schematically illustrates an example of a system that includes the certificate bridge device of FIG. 6 as an intermediary between one or more devices in a medical facility and one or more certificate authorities.

FIG. 8 schematically illustrates an example of a method of managing digital certificates for the medical device of FIG. 1.

### DETAILED DESCRIPTION

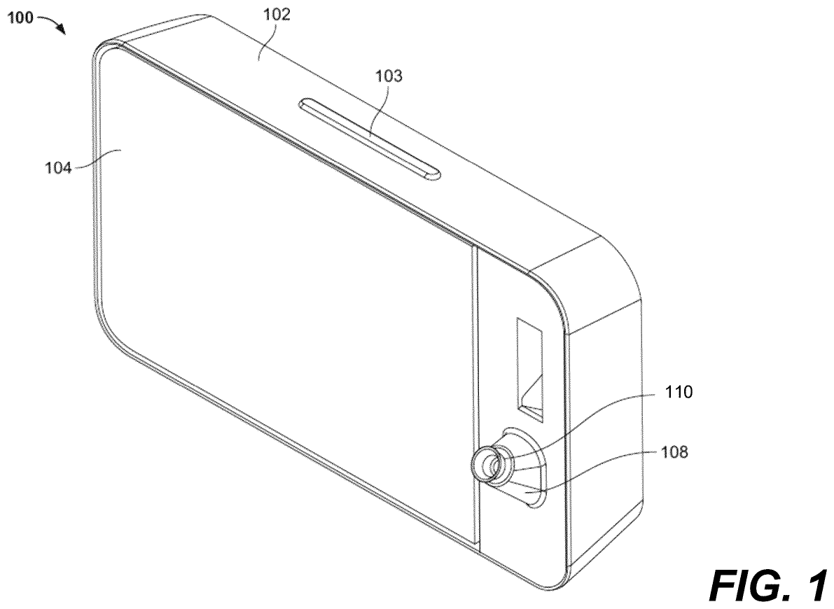

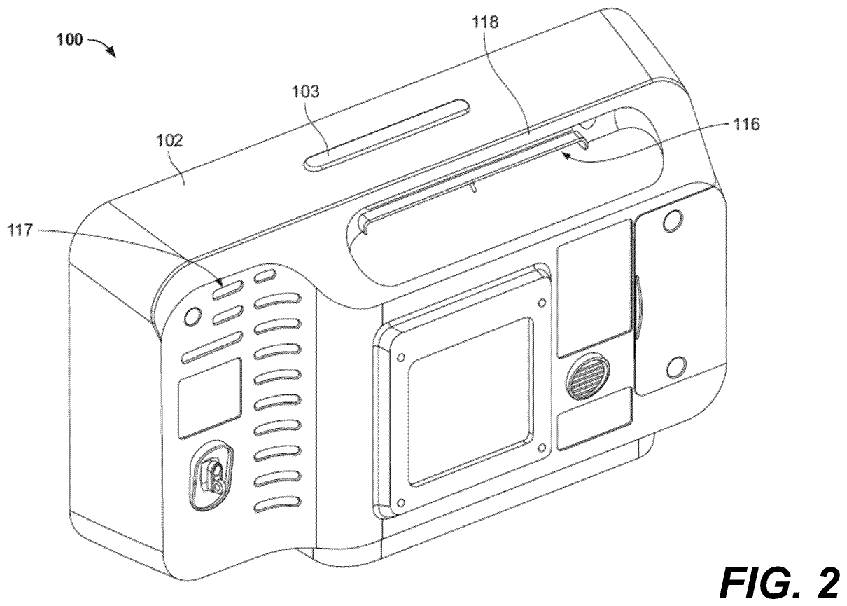

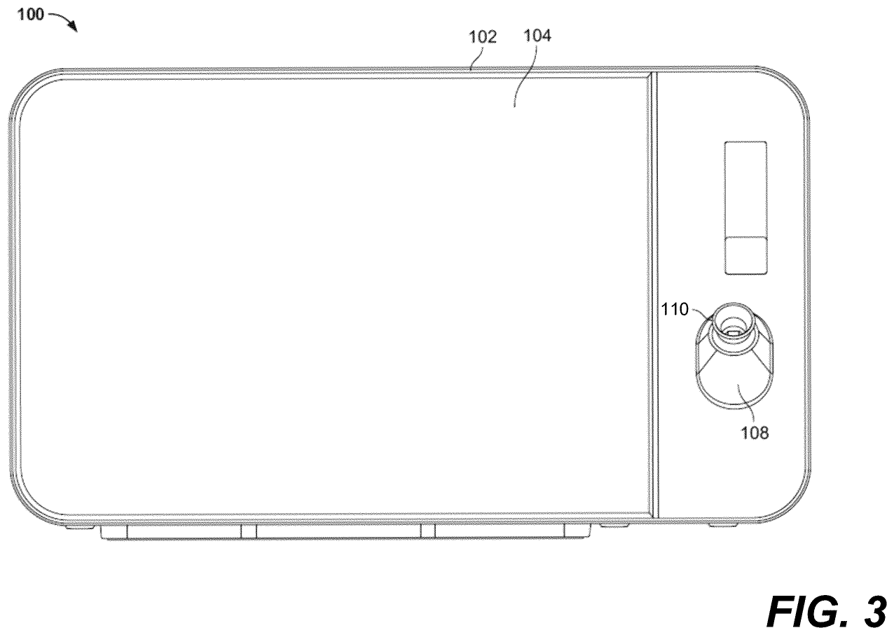

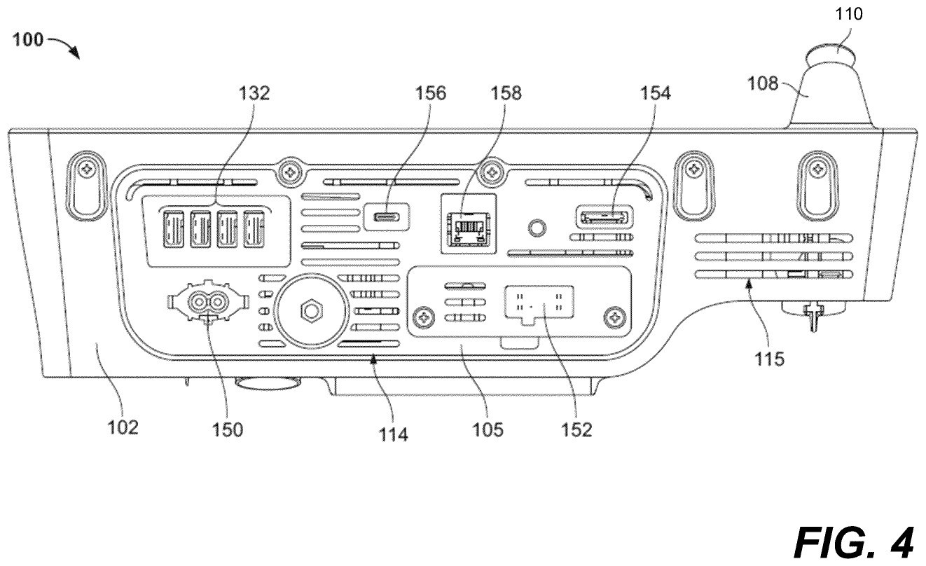

FIG. 1 is a front isometric view of a medical device 100 that can be used to monitor one or more physiological variables of a patient. FIG. 2 is a rear isometric view of the medical device 100. FIG. 3 is a front view of the medical device 100. FIG. 4 is a bottom view of the medical device 100. As shown in FIGS. 1-4, the medical device 100 includes a housing 102, and a display screen 104 mounted on a front portion of the housing 102. The display screen 104 is a touch sensitive touchscreen that receives inputs from a user of the medical device 100 such as a caregiver, a physician, a clinician, a nurse, or other trained healthcare professional.

The medical device 100 is designed for use in a clinical setting. For example, the medical device 100 can be used to continuously monitor one or more physiological variables of a patient admitted to a medical facility such as a hospital, medical clinic, and the like. Additionally, the medical device 100 can operate as a spot monitor for episodic monitoring of one or more physiological variables of a plurality of patients in the medical facility.

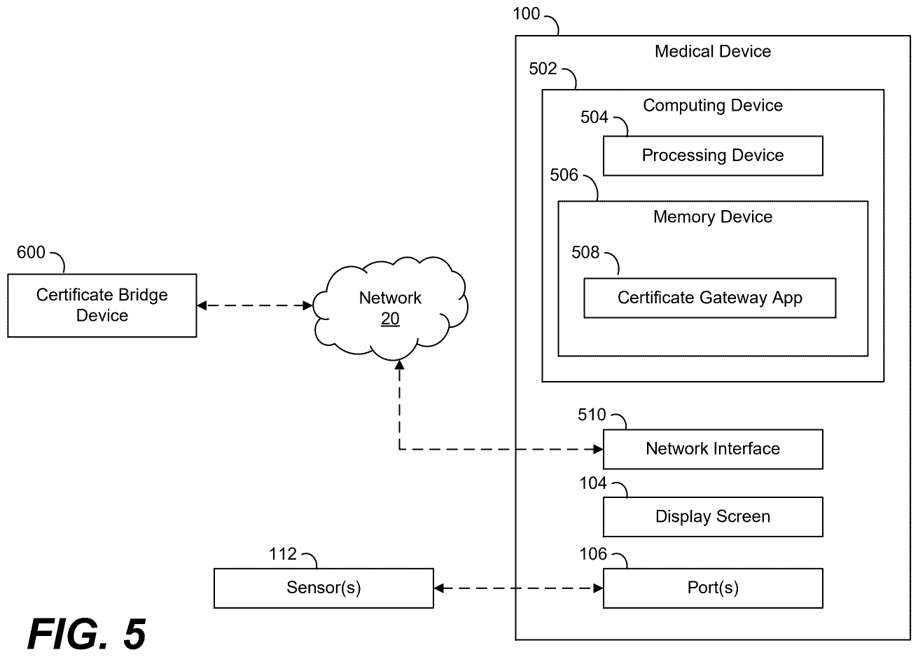

The medical device 100 is configured for connection to one or more sensors 112 (see FIG. 5) that can be used to measure the one or more physiological variables including, without limitation, blood pressure, blood oxygen saturation (SpO2), heart rate, pulse rate, body temperature, and respiration rate. The sensors 112 can include a thermometer module 108 to measure body temperature, a non-invasive blood pressure cuff to measure systolic and diastolic blood pressure, a pulse oximeter to measure blood oxygen saturation and pulse rate, and can include additional types of sensors 112 for measuring additional types of physiological variables.

The sensors 112 can include cables that terminate into connectors that plug into various ports included on the housing 102 of the medical device 100 for transmission of data to the medical device 100. As shown in FIG. 4, the housing 102 includes a first port 150 for connecting the non-invasive blood pressure cuff and a second port 152 for connecting the pulse oximeter. Optionally, the medical device 100 can include a third port 154 for connecting the medical device 100 to a printer. Also, the medical device 100 can include one or more universal serial bus (USB) ports 132, a USB-C port 156, and a registered jack (RJ) interface 158. In some examples, at least some of the sensors 112 wirelessly transmit data to the medical device 100 via a wireless connection established through Wi-Fi, Bluetooth, or another wireless protocol.

In further examples, the housing 102 includes a port for connection of a scanner to the medical device 100. The scanner can be used to scan machine-readable data such as a barcode including linear barcodes, quick-response (QR) codes, and the like that identify a person such as a patient who is being monitored by the medical device 100 or a user of the medical device 100 (e.g., a nurse, a doctor, a caregiver, and the like). The machine-readable data can further identify an object such as medical equipment or a medication being administered to the patient.

In the example shown in FIGS. 1-4, the thermometer module 108 is integrated with the medical device 100. The thermometer module 108 includes a port 110 for housing a handheld probe that can be orally inserted into the mouth of a patient to take a temperature reading. Alternatively, the housing 102 of the medical device 100 can be configured to house a handheld probe that can be inserted into an ear of the patient to take a temperature reading.

The medical device 100 receives data from the sensors 112 for processing. The medical device 100 generates and displays numerical values and/or waveforms based on the data received from the sensors 112 for monitoring the one or more physiological variables of the patient. The medical device 100 displays the numerical values and/or waveforms of the one or more physiological variables on the display screen 104. Also, the medical device 100 generates alarms when the data received from the sensors 112 indicates an alarm condition is detected.

The alarms generated by the medical device 100 can include a visual alarm displayed on the display screen 104 and an audible alarm emitted by a speaker of the medical device 100. An additional visual alarm can be displayed on an illumination unit 103, which is positioned on the top portion of the housing 102 of the medical device 100. The illumination unit 103 allows for distant viewing of critical alarms. The critical alarms generated on the illumination unit 103 can be viewed across a 360 degree field of view around the medical device 100. In some examples, both a visual alarm is displayed on the display screen 104 and a further visual alarm is generated on the illumination unit 103 to indicate a critical status of a detected alarm condition.

The illumination unit 103 can include one or more light-emitting diodes (LEDs) that emit light that is visible through a translucent cover. As an illustrative example, the illumination unit 103 can emit light having a color (e.g., yellow or red) based on a severity of the alarm. Additionally, or alternatively, the illumination unit 103 can emit light flashes based on a predetermined pattern associated with the severity of the alarm (e.g., an increased blinking rate to indicate severe conditions and a decreased blinking rate to indicate less severe conditions).

FIG. 5 schematically illustrates an example of the medical device 100. As shown in FIG. 5, the medical device 100 includes a computing device 502 having at least one processing device 504 and at least one memory device 506 that stores software instructions that, when executed by the at least one processing device 504, cause the at least one processing device 504 to perform the various aspects, functions, and operations described herein.

The at least one processing device 504 is an example of a processing unit such as a central processing unit (CPU). The at least one processing device 504 can include one or more CPUs. In some examples, the at least one processing device 504 includes one or more digital signal processors, field-programmable gate arrays, and/or other types of electronic circuits.

The at least one memory device 506 is an example of a computer-readable data storage device that operates to store data and instructions for execution by the at least one processing device 504. The at least one memory device 506 includes computer-readable media, which includes any media that can be accessed by the at least one processing device 504. The computer-readable media can include computer-readable storage media and computer-readable communication media. The computer-readable storage media includes volatile and nonvolatile, removable and non-removable media implemented in any device that can store information such as computer-readable instructions, data structures, program modules, or other data. The computer-readable storage media can include random access memory, read only memory, electrically erasable programmable read only memory, flash memory, and other memory technology, including any medium that can be used to store information that can be accessed by the at least one processing device 504. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

The at least one memory device 506 stores a certificate gateway application 508 that enables the medical device 100 to initiate a certificate enrollment via a trusted authentication and authorization protocol. Further, the certificate gateway application 508 enables the medical device 100 to renew the certificate enrollment after a predetermined period of time has elapsed after an initial certificate enrollment or a prior renewal of the certificate enrollment.

The medical device 100 includes a network interface 510 that facilitates connection to a network 20 that can include any type of wired or wireless connections or any combinations thereof. The network interface 510 can include wired interfaces and/or wireless interfaces. For example, the network interface 510 can be used to wirelessly connect the medical device 100 to the network 20 such as through Wi-Fi and the like. Alternatively, or additionally, the network interface 510 can be used to connect the medical device 100 to the network 20 using wired connections such as through Ethernet or Universal Serial Bus (USB) cables.

As further shown in FIG. 5, the one or more sensors 112 can plug into one or more ports 106 of the medical device 100 (e.g., the first port 150, the second port 152, and the like), and/or that can wirelessly transmit data to the medical device 100, and/or that can be integrated with the medical device 100 in accordance with the examples described above.

The medical device 100 communicates with a certificate bridge device 600 over the network 20. In some examples, the network 20 is a local, private network. The certificate bridge device 600 is locally installed in the facility where the medical device 100 is deployed such as a hospital, medical clinic, and the like. The certificate bridge device 600 receives a request for certificate enrollment or certificate renewal from the medical device 100. After receipt of the request, the certificate bridge device 600 authenticates the medical device 100, determines whether the medical device 100 is authorized to participate in the certificate enrollment or renewal, and validates the request for certificate enrollment or renewal.

The certificate bridge device 600 is further configured to build an endpoint request for a certificate authority to return a signed copy of a digital certificate (also known as a public key certificate or identity certificate). Upon receipt of the signed copy of the digital certificate from the certificate authority, the certificate bridge device 600 sends the signed copy of the digital certificate to the medical device 100 for storage in a secure location on the at least one memory device 506 of the medical device 100. Accordingly, the certificate bridge device 600 acts as an intermediary between the medical device 100 and the certificate authority.

The signed copy of the digital certificate carries cryptographic proof that the medical device 100 was in the control of a designated client and establishes trust in the details assigned by the client (i.e., a manufacturer-issued digital certificate or data template discussed below).

Once the medical device 100 receives the signed copy of the digital certificate from the certificate bridge device 600, the signed copy of the digital certificate allows the medical device 100 to access external systems and resources such as an electronic medical record (EMR) system that maintains a plurality of EMRs for a plurality of patients. The medical device 100 can further use the signed copy of the digital certificate to access additional external systems and resources such as overread systems, servers equipped with artificial intelligence, and the like. For example, the signed copy of the digital certificate allows the medical device 100 to access external resources by enabling the external resources to authenticate the medical device 100 and to confirm an identity of an owner or an operator of the medical device 100.

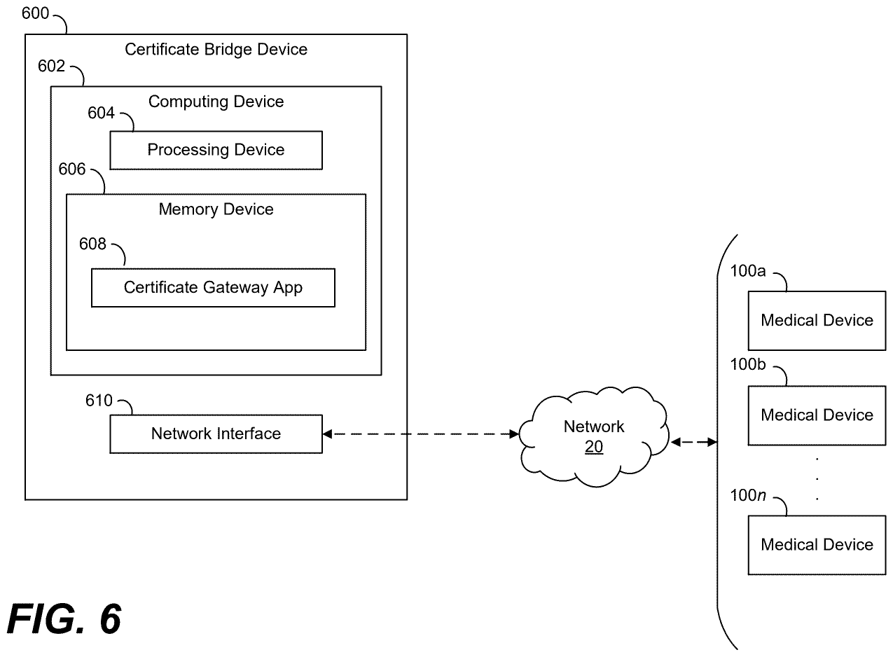

FIG. 6 schematically illustrates an example of the certificate bridge device 600 that facilitates digital certificate management for a plurality of devices within a facility, including the medical device 100. The certificate bridge device 600 facilitates distribution of digital certificates for the plurality of devices that are fielded in the facility. The certificate bridge device 600 establishes root of trust between the devices and one or more certificate authorities that manage the distribution of the digital certificates. The certificate bridge device 600 can work with devices in the field that are not equipped with a private key such as legacy devices.

The certificate bridge device 600 includes a computing device 602 having at least one processing device 604 and at least one memory device 606 that stores software instructions that, when executed by the at least one processing device 604, cause the at least one processing device 604 to perform the various aspects, functions, and operations described herein. In at least some aspects, the computing device 602 including the at least one processing device 604 and the at least one memory device 606 are similar to the computing device 502, the at least one processing device 504, and the at least one memory device 506 of the medical device 100.

The at least one processing device 604 is an example of a processing unit such as a central processing unit (CPU). The at least one processing device 604 can include one or more CPUs. In some examples, the at least one processing device 604 includes one or more digital signal processors, field-programmable gate arrays, and/or other types of electronic circuits.

The at least one memory device 606 is an example of a computer-readable data storage device that operates to store data and instructions for execution by the at least one processing device 604. The at least one memory device 606 includes computer-readable media, which includes any media that can be accessed by the at least one processing device 604. The computer-readable media can include computer-readable storage media and computer-readable communication media. The computer-readable storage media includes volatile and nonvolatile, removable and non-removable media implemented in any device that can store information such as computer-readable instructions, data structures, program modules, or other data. The computer-readable storage media can include random access memory, read only memory, electrically erasable programmable read only memory, flash memory, and other memory technology, including any medium that can be used to store information that can be accessed by the at least one processing device 504. The computer-readable storage media is non-transitory.

The computer-readable communication media embodies computer-readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. The computer-readable communication media can include wired media such as a wired network or direct-wired connection, and wireless media such as acoustic, radio frequency, infrared, and other wireless media. Combinations of any of the above are within the scope of computer-readable media.

As shown in FIG. 6, the at least one memory device 606 stores a certificate gateway application 608 which communicates with the certificate gateway application 508 installed on the medical device 100. The certificate gateway application 608 is a guest application installed on the certificate bridge device 600 which is deployed locally, on-premises of the medical facility where the plurality of medical devices are deployed in the field. The certificate gateway application 608, which is locally installed, provides a mechanism for the medical devices to initiate a certificate enrollment via a trusted authentication and authorization protocol.

The protocol can either be a certificate signing request (CSR) from a medical device that is already provisioned with a digital certificate or basic authentication from a medical device that has been provided with an appropriate data template. After the medical device is authenticated and authorized with the certificate gateway application 608, the certificate gateway application 608 constructs an endpoint request, which sends a CSR to one of the supporting certificate authorities (e.g., ADCS, Keyfactor, etc.). The certificate authority returns a signed copy of a digital certificate to the certificate gateway application 608. The certificate gateway application 608 then distributes the signed copy of the digital certificate to the requesting device.

The certificate gateway application 608 eliminates the need for physical access to each device within a facility with a trusted median to provision each device. Instead, the certificate gateway application 608 advantageously provides a remote means to facilitate certificate management for a plurality of devices in the field. Remote digital certificate provisioning for fleet management is a significant improvement over workflows that require human intervention at each device of a plurality of devices in the field because it reduces time (e.g., about 10 minutes per device) and also reduces potential sources of human error.

The certificate gateway application 608 enables the certificate bridge device 600 to receive a request for certificate enrollment or renewal from the medical device 100. When the medical device 100 is provisioned with a manufacturer-issued digital certificate, the certificate gateway application 608 utilizes the manufacturer-issued digital certificate from the medical device 100 to authenticate the medical device 100 and to build the endpoint request for a certificate authority to provide a signed copy of the digital certificate.

The manufacturer-issued digital certificate carries cryptographic proof of manufacture. For example, the manufacturer-issued digital certificate establishes an identity of the manufacturer, a type of medical device (e.g., spot monitor), a device serial number, and a timestamp of issuance. The manufacturer-issued digital certificate is used by the certificate bridge device 600 to authenticate the medical device 100.

An endpoint that offers services to a specific client is not be able to discriminate between the medical devices 100 based on the manufacturer-issued digital certificates. Instead, the endpoint is only able to establish authenticity (i.e., the medical device 100 was manufactured by a certain entity). Given the foregoing, a signed copy of a digital certificate from a certificate authority (as will be described further below) is necessary to authenticate that the medical device 100 was, at the time of issuance of the signed copy of the digital certificate, in control of the client. Once authenticated, the medical device 100 is able to access the endpoint.

Alternatively, when the medical device 100 is not provisioned with a manufacturer-issued digital certificate, the certificate gateway application 608 can utilize a data template from the medical device 100. In some examples, the data template can include a secure hash sum of manufacturer data, customer data, and time sensitive data to authenticate the medical device 100. The data template can further be used to build an endpoint request for the medical device 100 for the certificate authority to provide the signed copy of the digital certificate.

The certificate gateway application 608 authenticates the medical device 100, determines whether the medical device 100 is authorized to participate in the certificate enrollment or renewal, and validates the request for certificate enrollment or renewal. Once the medical device 100 is authenticated and the certificate enrollment/renewal request is validated, the certificate gateway application 608 builds an endpoint request for a certificate authority to return a signed copy of the digital certificate. Upon receipt of the signed copy of the digital certificate from the certificate authority, the certificate bridge device 600 sends the signed copy of the digital certificate to the medical device 100 for storage in a secure location on the at least one memory device 506 of the medical device 100. Accordingly, the certificate bridge device 600 acts as an intermediary between the medical device 100 and the certificate authority.

The certificate bridge device 600 includes a network interface 610 that facilitates connection to the network 20 that can include any type of wired or wireless connections or any combinations thereof. The network interface 610 can include wired interfaces and/or wireless interfaces. For example, the network interface 610 can be used to wirelessly connect the certificate bridge device 600 to the network 20 such as through Wi-Fi and the like. Alternatively, or additionally, the network interface 610 can be used to connect the certificate bridge device 600 to the network 20 using wired connections such as through Ethernet or USB cables.

As shown in FIG. 6, the certificate bridge device 600 can communicate with a plurality of medical devices 100a, 100b . . . 100n over the network 20 in the medical facility. The plurality of medical devices can include devices that are similar to the monitoring device shown in FIGS. 1-4, and/or can include additional types of medical devices such as hospital beds, infusion pumps, and the like. Accordingly, the certificate bridge device 600 can be used to obtain signed copies of digital certificates for a plurality of medical devices in a medical facility.

The certificate bridge device 600 automates the process of obtaining and forwarding the signed copies of the digital certificates to significantly reduce the amount of time that would otherwise be required to obtain the signed copies of the digital certificates for the plurality of medical devices. For example, the process of obtaining the signed copies of the digital certificates is typically performed manually by use of a portable authentication device (e.g., USB flash drive, thumb drive, memory stick, pen drive, etc.) that is manually inserted into each medical device of the plurality of medical devices. The certificate bridge device 600 automatically receives requests for digital certificate enrollment or renewal, generates the endpoint request for the certificate authority, and sends the signed copies of the digital certificates to the plurality of medical devices 100 without manual intervention.

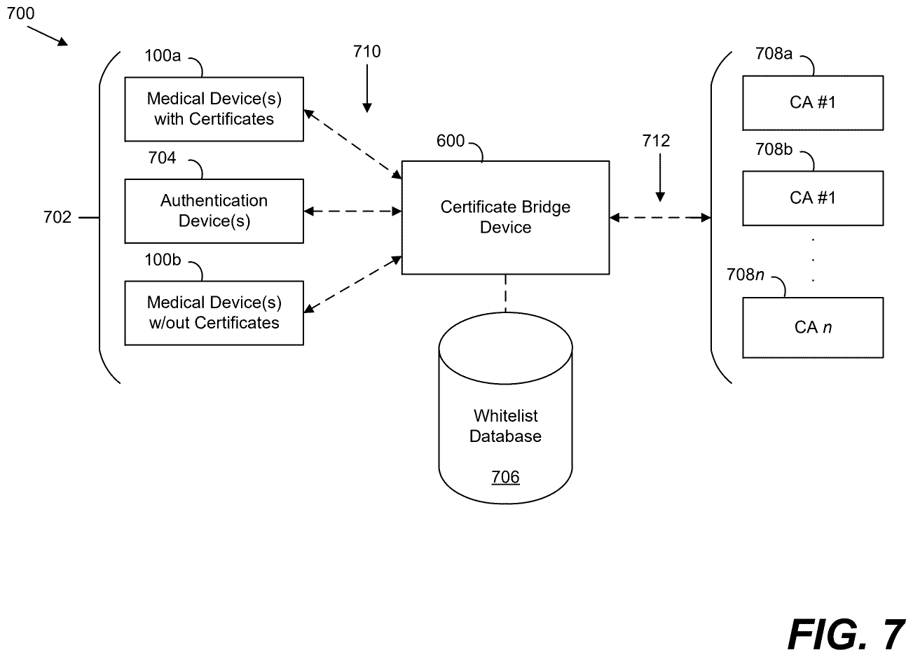

FIG. 7 schematically illustrates an example of a system 700 that includes the certificate bridge device 600 as an intermediary between one or more devices 702 in a medical facility and one or more certificate authorities 708. The one or more devices 702 include medical devices 100a that have installed thereon a manufacturer-issued certificate.

The manufacturer-issued certificate is generated by a cryptographic tool for installation on a secure portion of the at least one memory devices 506 of the medical devices 100a. The manufacturer-issued certificate is installed during manufacture of the medical devices 100a. The medical devices 100a can communicate with the certificate bridge device 600 via an Enrollment over Secure Transport (EST) protocol or other types of protocols.

The manufacturer-issued certificate can be used to generate a certificate signing request (CSR), which is an intermediate step in the process of obtaining a signed copy of a digital certificate from a certificate authority. To generate the CSR, the medical devices 100a each generate a pair of values (i.e., public and private keys), fill in details of the CSR (e.g., the subject name), sign the CSR with the private key, and combine the CSR with the public key. The medical devices 100a then send the CSR to the certificate bridge device 600.

The one or more devices 702 further include authentication devices 704 that are used to protect access to medical devices and other types of devices, networks, and online services. The authentication devices 704 can include USB flash drives, thumb drives, memory sticks, pen drives, and the like that are manually inserted into devices fielded in the facility. The authentication devices 704 can support one-time passwords (OTP), public-key cryptography, authentication, and the Universal 2nd Factor (U2F) and FIDO2 protocols. The authentication devices 704 can utilize the certificate bridge device 600 to renew their digital certificates.

The one or more devices 702 include medical devices 100b that do not have a manufacturer-issued certificate. Instead, the medical devices 100b include a data template that includes parameters such as common name (CN), domain component (DC), organizational unit (OU), and the like. A secure hash sum can be generated from the data template. In some examples, the secure hash sum includes manufacturer data, customer data, and time sensitive data to provide a rolling code (also known as a hopping code) to prevent replay attacks.

As further shown in FIG. 7, the system 700 includes a whitelist database 706 that identifies all devices in the facility that are authorized to utilize the certificate bridge device 600 to obtain a signed copy of a digital certificate. For example, some of the one or more devices 702 may be included in the whitelist database 706 while other devices are not. The whitelist database 706 can include a listing of serial numbers and/or MAC addresses of the devices authorized to utilize the certificate bridge device 600 to obtain signed copies of digital certificates.

The certificate bridge device 600 is in communication with one or more certificate authorities 708. For example, the certificate bridge device 600 can communicate with Active Directory Certificate Service (ADCS), Keyfactor (i.e., EJBCA (Enterprise JavaBeans Certificate Authority)), and Certificate Management (CM) REST to issue and manage public key infrastructure (PKI) certificates used in secure communication and authentication protocols.

The certificate bridge device 600 communicates with the one or more devices 702 over a first communication channel 710. The certificate bridge device 600 further communicates with the one or more certificate authorities 708 over a second communication channel 712. In some examples, the first communication channel 710 and the second communication channel 712 utilize the same protocol. Alternatively, the first communication channel 710 and the second communication channel 712 utilize different protocols.

The second communication channel 712 is determined based on the certificate authority 708 which the certificate bridge device 600 communicates with. For example, when communicating with a client-controlled ADCS, the certificate bridge device 600 can use a Microsoft® proprietary protocol for ADCS. When communicating with a client-controlled KeyFactor (or other service), the certificate bridge device 600 can use an Enrollment over Secure Transport (EST) protocol to communicate to an instance of a KeyFactor EJBCA server. Accordingly, the certificate bridge device 600 utilizes an appropriate protocol for the second communication channel 712 based on the type of certificate authority 708.

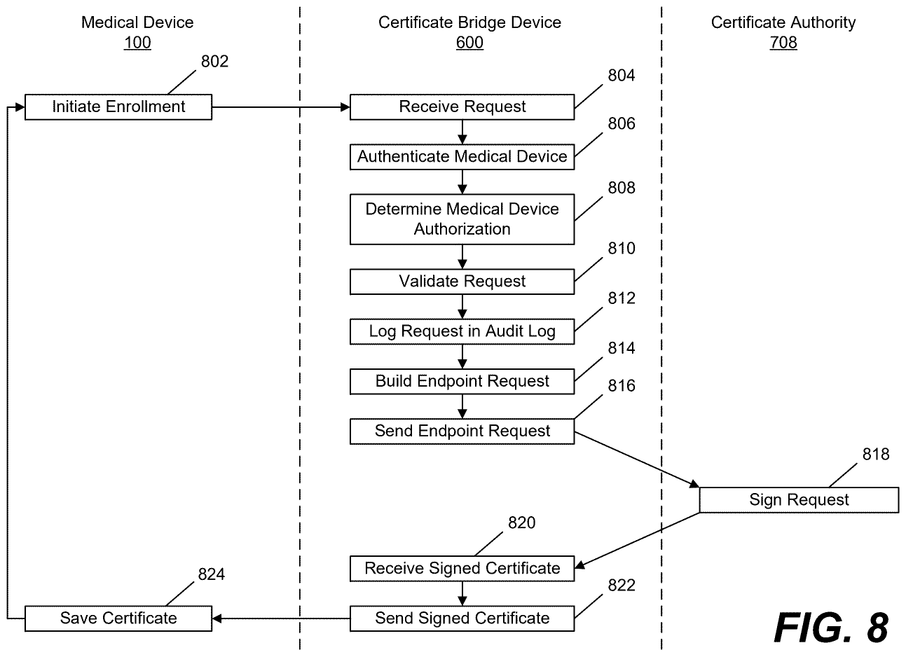

FIG. 8 schematically illustrates an example of a method 800 of managing digital certificates for the medical device 100. At least some aspects of the method 800 are performed by the certificate gateway application 608 installed on the certificate bridge device 600.

The method 800 includes an operation 802 of initiating digital certificate enrollment or renewal on the medical device 100. In some examples, the digital certificate enrollment or renewal is initiated based on an elapse of time such as a predetermined amount of time that has elapsed from a prior digital certificate enrollment or renewal. Alternatively, the digital certificate enrollment or renewal can be initiated based on usage such as quantity of times that a prior digital certificate issued to the medical device 100 has been used. The digital certificate enrollment or renewal can be initiated by the certificate gateway application 508 installed on the medical device 100. Alternatively, the digital certificate enrollment or renewal can be initiated by the certificate gateway application 608 installed on the certificate bridge device 600.

The method 800 includes an operation 804 of receiving a request from the medical device 100. In some examples, the request from the medical device 100 is an unsigned certificate signing request (CSR). Alternatively, the request from the medical device 100 can include a data template in examples where the medical device 100 does not possess a private key. In some examples, a secure hash sum of manufacturer data, customer data, and time sensitive data is generated from the data template. In some examples, the secure hash sum derived from the data template provides a rolling code (also known as a hopping code) to prevent replay attacks.

The method 800 includes an operation 806 of authenticating the medical device 100. In some examples, operation 806 includes performing mutual TLS (mTLS), which is a type of authentication in which the medical device 100 and the certificate bridge device 600) authenticate each other using the transport layer security (TLS) protocol.

The method 800 includes an operation 808 of determining whether the medical device 100 is authorized to send the request. Operation 808 can include checking the whitelist database 706 to determine whether the medical device 100 is authorized. Operation 808 can include looking up the serial number, MAC address, or other identifier of the medical device 100 in the whitelist database 706 to confirm the medical device 100 is authorized to send the request.

The method 800 includes an operation 810 of validating the request received from the medical device 100 in operation 804. For example, operation 810 can include checking an expiration date of the request to confirm that the request has not expired. Operation 810 can also include checking whether one or more rules of the request are satisfied. For example, operation 810 can include looking at the fields of the CSR to determine validity (and how to process it). The one or more rules can include matching the subject name to ensure it complies with the organization (e.g., matches a pattern such as CN=C360-XXXXXX, DC=customer, DC=com), checking a starting date to ensure it is not within some tolerance of current time, and checking an ending date to ensure it complies with the policy configured into certificate gateway application 608. Pattern checking can be accomplished using regular expressions allowing for sophisticated checks. Further, the validation checking can also allow the certificate bridge device 600 to direct the request to the certificate authority 708 (see operation 816).

The method 800 includes an operation 812 of logging the request received from the medical device 100 into an audit log. In some examples, the audit log is encrypted. The audit log provides a timeline of requests received by the certificate bridge device 600. For example, the audit log can record an event that occurred (i.e., the request received in operation 804), a time at which the request was received, the medical device 100 responsible for the request.

The method 800 includes an operation 814 of building an endpoint request that is used by the certificate bridge device 600 to request a signed copy of a digital certificate. The endpoint request includes the request received from the medical device 100 in operation 804, and further includes facility credentials of the facility where the medical device 100 is deployed.

The endpoint request is protocol specific based on the certificate authority 708. As an illustrative example, the endpoint request can have a structure that follows the EST protocol which uses Hypertext Transfer Protocol (HTTP) to exchange information. In such examples, the endpoint request follows the HTTP POST standard.

The method 800 includes an operation 816 of sending the endpoint request to a certificate authority 708. Operation 816 can include sending the endpoint request to active directory certificate services (AD CS), Keyfactor, and other certificate authorities.

The method 800 includes an operation 818 of having the certificate authority 708 sign the request. Operation 818 can be performed in accordance with standard certificate authority procedures. For example, the certificate authority 708 evaluates the request received from the certificate bridge device 600 and signs the request using its private key. Accordingly, the certificate authority 708 stores, signs, and issues a signed copy of the digital certificate that contains a public key, a cryptographic signature, and other information.

The method 800 includes an operation 820 of receiving a signed copy of the request from the certificate authority 708. In some examples, the signed copy of the request is an X.509 certificate that binds an identity to a public key using a digital signature. For example, the X.509 certificate contains an identity of the facility where the medical device 100 is deployed (e.g., a hostname, an organization, or an individual) and a public key signed by the certificate authority.

In some examples, operation 820 can include auditing the signed copy of the request from the certificate authority. For example, operation 820 can include consulting the audit log to confirm that the signed copy is received as a result of the request validated in operation 810.

The method 800 includes an operation 822 of sending the signed copy of the request to the medical device 100. The method 800 includes an operation 824 of having the medical device 100 save the signed copy of the request in a secure location on the at least one memory device 506. After completion of operation 824, the method 800 can return to operation 802 to initiate a new digital certificate enrollment or renewal on the medical device 100 such as after a predetermined amount of time from a prior digital certificate enrollment or renewal has elapsed.

The various embodiments described above are provided by way of illustration only and should not be construed to be limiting in any way. Various modifications can be made to the embodiments described above without departing from the true spirit and scope of the disclosure.

What is claimed is:

1. A device for managing digital certificates for a medical device, the device comprising:
at least one processing device; and
at least one memory device storing software instructions that, when executed by the at least one processing device, cause the at least one processing device to:
receive a request from the medical device;
authenticate the medical device;
determine the medical device is authorized to send the request;
validate the request from the medical device;
build an endpoint request that includes:
the request from the medical device; and
facility credentials of a facility where the medical device is deployed;
send the endpoint request to a certificate authority;
receive a signed copy of the request from the certificate authority; and
send the signed copy of the request to the medical device.

2. The device of claim 1, wherein validate the request includes confirmation of an expiration date of the request and satisfaction of one or more rules.

3. The device of claim 1, wherein the instructions, when executed by the at least one processing device, further cause the at least one processing device to:
log the request from the medical device into a secure audit log.

4. The device of claim 1, wherein determine the medical device is authorized to send the request is based on whether the medical device is included in a whitelist database.

5. The device of claim 1, wherein the request from the medical device includes an unsigned certificate signing request.

6. The device of claim 1, wherein the request from the medical device includes a secure hash sum of manufacturer data, customer data, and time sensitive data.

7. A method of managing digital certificates for a medical device, the method comprising:
receiving a request from the medical device;
authenticating the medical device;
determining the medical device is authorized to send the request;
validating the request from the medical device;
building an endpoint request that includes:
the request from the medical device; and
facility credentials of a facility where the medical device is deployed;
sending the endpoint request to a certificate authority;
receiving a signed copy of the request from the certificate authority; and
sending the signed copy of the request to the medical device.

8. The method of claim 7, wherein validating the request includes checking for an expiration date of the request and confirming one or more rules are satisfied.

9. The method of claim 7, further comprising:
logging the request from the medical device into a secure audit log.

10. The method of claim 7, wherein the request from the medical device includes an unsigned certificate signing request.

11. The method of claim 7, wherein the request from the medical device includes a secure hash sum of manufacturer data, customer data, and time sensitive data.

12. An authentication system comprising:
a medical device; and
a certificate bridge device configured to:
receive a request from the medical device;
generate an endpoint request that includes the request from the medical device and facility credentials of a facility where the medical device is deployed;
send the endpoint request to a certificate authority;
receive a signed copy of the request from the certificate authority; and
send the signed copy of the request to the medical device.

13. The system of claim 12, wherein the certificate bridge device communicates with the medical device over a first communication channel; and
wherein the certificate bridge device communicates with the certificate authority over a second communication channel.

14. The system of claim 13, wherein the first communication channel and the second communication channel have different communications protocols.

15. The system of claim 13, wherein the first communication channel and the second communication channel share a communications protocol.

16. The system of claim 12, wherein the certificate bridge device is an intermediary between the medical device and the certificate authority.

17. The system of claim 12, wherein the certificate bridge device sends the signed copy of the request to the medical device without manual intervention.

18. The system of claim 12, wherein the certificate bridge device is locally installed in the facility where the medical device is deployed.

19. The system of claim 12, wherein the request from the medical device is an unsigned certificate signing request.

20. The system of claim 12, wherein the request from the medical device includes a secure hash sum of manufacturer data, customer data, and time sensitive data.
